(19) United States
(12) Patent Application Publication
(10) Pub. No.: US 2025/0255534 A1
(43) Pub. Date: Aug. 14, 2025

(54) PVC EPISODE DETECTION AND STORAGE

(71) Applicant: Cardiac Pacemakers, Inc., St Paul, MN (US)

(72) Inventors: Krzysztof Z. Siejko, Maple Grove, MN (US); Keith L. Herrmann, Minneapolis, MN (US); Abhijit Rajan, Shoreview, MN (US)

(21) Appl. No.: 19/045,986

(22) Filed: Feb. 5, 2025

Related U.S. Application Data
(60) Provisional application No. 63/551,516, filed on Feb. 8, 2024.

(51) Int. Cl. A61B 5/333; A61B 5/00; A61B 5/02; A61B 5/287; A61B 5/346

(52) U.S. Cl. CPC A61B 5/333; A61B 5/0006; A61B 5/0031; A61B 5/02028; A61B 5/287; A61B 5/346; A61B 5/686

(57) ABSTRACT

A method includes detecting that a first premature ventricular contraction (PVC) episode occurred within a first length of electrocardiogram (ECG) data. The method further includes recording the first length of ECG data to memory in response to the detecting. A second PVC episode is determined to have occurred after the first PVC episode and within a second length of ECG data, and the second PVC episode is determined to be either a first type of PVC episode or a second type of PVC episode. The method further includes determining whether to either overwrite the first length of ECG data or to separately record the second length of ECG data to the memory based, at least in part, on the determining that the second PVC episode is the first type of PVC episode or the second type of PVC episode.

### CROSS REFERENCE TO RELATED APPLICATION

This application claims priority to Provisional Application No. 63/551,516, filed Feb. 8, 2024, which is herein incorporated by reference in its entirety.

### TECHNICAL FIELD

Instances of the present disclosure relate generally to approaches for efficient recording of cardiac activity data involving premature ventricular contractions.

### BACKGROUND

Medical devices that allow physicians to monitor cardiac activity are becoming increasingly common in diagnosing and treating medical conditions in patients. Cardiac monitoring can be used, for example, to identify abnormal cardiac rhythms, so that critical alerts can be provided to patients, physicians, or other care providers and so that patients can be treated as needed.

### SUMMARY

In Example 1, a method includes detecting that a first premature ventricular contraction (PVC) episode occurred within a first length of electrocardiogram (ECG) data. The first PVC episode is a first type of PVC episode. The method further includes, in response to the detecting, recording the first length of ECG data to memory. A second PVC episode is determined to have occurred after the first PVC episode and within a second length of ECG data, and the second PVC episode is determined to be either the first type of PVC episode or a second type of PVC episode. The method further includes determining whether to either overwrite the first length of ECG data or to separately record the second length of ECG data to the memory based, at least in part, on the determining that the second PVC episode is the first type of PVC episode or the second type of PVC episode.

In Example 2, the method of Example 1, further includes determining that the second PVC episode is the first type of PVC episode and, in response, overwriting the first length of ECG data with the second length of ECG data.

In Example 3, the method of any of Examples 1 or 2, wherein the first type of PVC episode and the second type of PVC episode are maximum burden episodes, wherein the first PVC episode is associated with a first burden value, wherein the second PVC episode is associated with a second burden value, wherein the first length of ECG data is overwritten by the second length of ECG data in response to the second burden value being greater than the first burden value.

In Example 4, the method of Example 3, further includes detecting that a third PVC episode occurred after the second PVC episode and within a third length of ECG data. The third PVC episode is associated with a third burden value. The method further includes overwriting the second length of ECG data with the third length of ECG data after determining that the third burden value is greater than the second burden value.

In Example 5, the method of Examples 3 or 4, wherein the first burden value and the second burden value are based on a number of PVCs within a predetermined time period.

In Example 6, the method of any of Examples 1 or 2, wherein the first type of PVC episode and the second type of PVC episode are couplet events and/or triplet events, wherein the first PVC episode is associated with a first number of couplets or triplets, wherein the second PVC episode is associated with a second number of couplets or triplets, wherein the first length of ECG data is overwritten by the second length of ECG data in response to the second number of couplets or triplets being greater than the first number of couplets or triplets.

In Example 7, the method of Example 1, wherein the first type and the second type are different types of PVC episodes. The method further includes determining that the second PVC episode is the second type of PVC episode and, in response, recording the second length of ECG data to the memory such that the memory contains both the first length of ECG data and the second length of ECG data.

In Example 8, the method of Example 7, wherein the first type of PVC episode is a maximum burden episode, wherein the second type of PVC episode is a couplet episode or a triplet episode.

In Example 9, the method of any of Examples 1-8, wherein the detecting that the first PVC episode occurred is based, at least in part, on a minimum PVC threshold being met.

In Example 10, the method of any of Examples 1-9, further including temporarily storing the first length of ECG data to a buffer before recording the first length to the memory.

In Example 11, the method of Example 10, wherein the ECG data not recorded to the memory is permanently deleted.

In Example 12, the method of any of Examples 1-11, further including wirelessly transmitting any PVC episodes recorded to the memory to a remote computing device.

In Example 13, a computer program product comprising instructions to cause one or more processors to carry out the steps of the method of Examples 1-12.

In Example 14, a computer-readable medium having stored thereon the computer program product of Example 13.

In Example 15, an implantable medical device comprising the computer-readable medium of Example 14.

In Example 16, a medical device includes a housing and electrodes coupled to the housing. The electrodes are for sensing cardiac activation signals used to generate ECG data. The medical device further includes memory and one or more processors programmed with instructions to cause the medical device to: (1) detect that a first PVC episode occurred within a first length of ECG data, wherein the first PVC episode is a first type of PVC episode; (2) in response to the detecting, record the first length of ECG data to the memory; (3) detect that a second PVC episode occurred after the first PVC episode and within a second length of ECG data; (4) determine that the second PVC episode is either the first type of PVC episode or a second type of PVC episode; and (5) determine whether to either overwrite the first length of ECG data or to separately record the second length of ECG data to the memory based, at least in part, on the second PVC episode being the first type of PVC episode or the second type of PVC episode.

In Example 17, the medical device of Example 16, further includes a buffer, wherein the first length of ECG data is temporarily stored to the buffer before being recorded to the memory.

In Example 18, the medical device of Example 17, wherein the ECG data temporarily stored to the buffer but not recorded to the memory is permanently deleted.

In Example 19, the medical device of Example 16, wherein the one or more processors are programmed with the instructions to further cause the medical device to determine that the second PVC episode is the first type of PVC episode and, in response, overwrite the first length of ECG data with the second length of ECG data.

In Example 20, the medical device of Example 16, wherein the first type of PVC episode and the second type of PVC episode are maximum burden episodes, wherein the first PVC episode is associated with a first burden value, wherein the second PVC episode is associated with a second burden value, wherein the first length of ECG data is overwritten by the second length of ECG data in response to the second burden value being greater than the first burden value.

In Example 21, the medical device of Example 20, wherein the first burden value and the second burden value are based on a number of PVCs within a predetermined time period.

In Example 22, the medical device of Example 16, wherein the first type of PVC episode and the second type of PVC episode are couplet events and/or triplet events, wherein the first PVC episode is associated with a first number of couplets or triplets, wherein the second PVC episode is associated with a second number of couplets or triplets, wherein the first length of ECG data is overwritten by the second length of ECG data in response to the second number of couplets or triplets being greater than the first number of couplets or triplets.

In Example 23, the medical device of Example 16, wherein the first PVC episode is detected based, at least in part, on a minimum PVC threshold being met.

In Example 24, the medical device of Example 16, wherein the one or more processors are programmed with the instructions to further cause the medical device to wirelessly transmit, at periodic intervals, any PVC episodes recorded to the memory to a remote computing device.

In Example 25, the medical device of Example 16, wherein the memory comprises a longer-term memory and a temporary memory, wherein the first length of ECG data is first recorded to the temporary memory and then to the longer-term memory.

In Example 26, the medical device of Example 25, wherein the longer-term memory has a greater storage capacity than the storage capacity of the temporary memory.

In Example 27, the medical device of Example 25, wherein the longer-term memory is flash memory, wherein the temporary memory is random-access memory.

In Example 28, the medical device of Example 25, wherein the temporary memory is a first-in first-out (FIFO) memory.

In Example 29, a method includes detecting that a first PVC episode occurred within a first length of ECG data. The first PVC episode is a first type of PVC episode. In response to the detecting, the first length of ECG data is recorded to memory. The method further includes detecting that a second PVC episode occurred after the first PVC episode and within a second length of ECG data, determining that the second PVC episode is either the first type of PVC episode or a second type of PVC episode, and determining whether to either overwrite the first length of ECG data or to separately record the second length of ECG data to the memory based, at least in part, on the determining that the second PVC episode is the first type of PVC episode or the second type of PVC episode.

In Example 30, the method of Example 29, further includes determining that the second PVC episode is the first type of PVC episode and, in response, overwriting the first length of ECG data with the second length of ECG data.

In Example 31, the method of Example 29, wherein the first type of PVC episode and the second type of PVC episode are maximum burden episodes, wherein the first PVC episode is associated with a first burden value, wherein the second PVC episode is associated with a second burden value, wherein the first length of ECG data is overwritten by the second length of ECG data in response to the second burden value being greater than the first burden value.

In Example 32, the method of Example 31, wherein the first burden value and the second burden value are based on a number of PVCs within a predetermined time period.

In Example 33, the method of Example 29, wherein the first type of PVC episode and the second type of PVC episode are couplet events and/or triplet events, wherein the first PVC episode is associated with a first number of couplets or triplets, wherein the second PVC episode is associated with a second number of couplets or triplets, wherein the first length of ECG data is overwritten by the second length of ECG data in response to the second number of couplets or triplets being greater than the first number of couplets or triplets.

In Example 34, the method of Example 29, wherein the first type and the second type are different types of PVC episodes. The method further includes determining that the second PVC episode is the second type of PVC episode and, in response, recording the second length of ECG data to the memory such that the memory contains both the first length of ECG data and the second length of ECG data.

In Example 35, the method of Example 34, wherein the first type of PVC episode is a maximum burden episode, wherein the second type of PVC episode is a couplet episode or a triplet episode.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a system that includes a medical device, in accordance with certain instances of the present disclosure.

FIGS. 2-4 show different views of a medical device, in accordance with certain instances of the present disclosure.

FIG. 5 shows a block diagram depicting an illustrative method, in accordance with certain instances of the disclosure.

FIG. 6 shows a strip of ECG data, in accordance with certain instances of the disclosure.

FIG. 7 is a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Medical devices can be equipped with one or more sensing components (e.g., sensors, electrodes) and programmed to sense physiological data such as electrocardiogram (ECG) data. To collect physiological data, one or more medical devices (e.g., implantable cardiac monitors/recorders, external cardiac monitors/recorders) can be implanted in or coupled to the patient such that the medical devices can sense the physiological data. Although such medical devices may attempt to continuously sense physiological data such as ECG data, to save power and memory capacity the medical devices may not ultimately record and then transmit all the sensed physiological data outside of the medical devices. Instead, the sensed physiological data may only be recorded and then transmitted in certain situations. For example, the medical devices may be programmed to process and analyze the sensed physiological data to determine the occurrence of certain health events such as episodes containing premature ventricular contractions (PVCs). If it is determined that PVCs have occurred, certain sensed physiological data can be recorded to longer-term memory of the medical device and then transmitted to another device. However, recording and transmitting physiological data every time a PVC occurs can be an inefficient use of the medical device's power and memory capacity.

Certain instances of the present disclosure are accordingly directed to approaches for prioritizing which cardiac events involving PVCs are recorded and ultimately transmitted for review.

### Cardiac Event Evaluation System

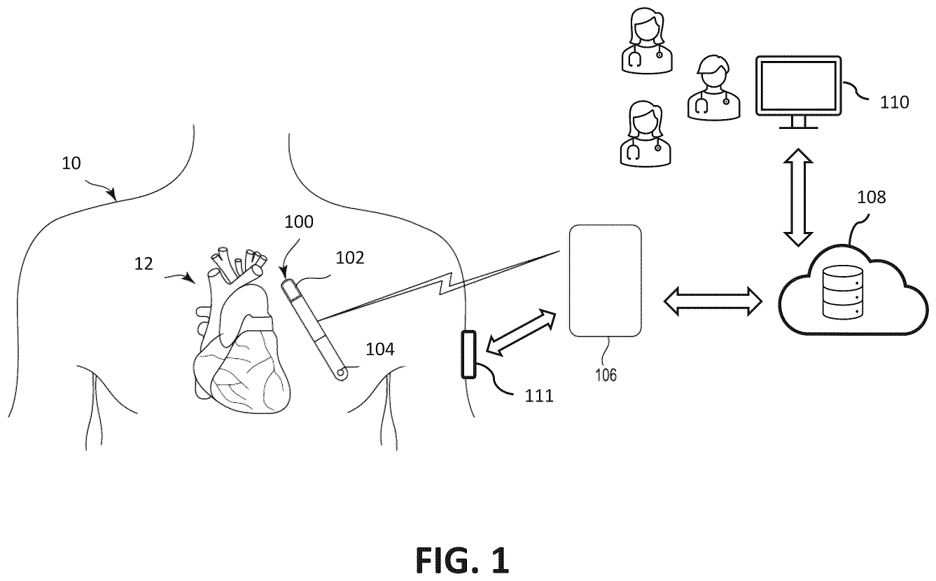

FIG. 1 is a schematic illustration of a medical device 100 within a cardiac event evaluation system. The medical device 100 can be implanted subcutaneously within an implantation location or pocket in the patient's chest or abdomen and may be configured to sense physiological signals associated with the patient's heart 12. The medical device 100 may be an implantable cardiac monitor (e.g., an implantable diagnostic monitor, an implantable loop recorder) configured and programmed to record physiological parameters such as, for example, one or more cardiac activation signals, heart sounds, blood pressure measurements, oxygen saturations. Although the medical device 100 of FIG. 1 is shown as an implantable cardiac monitor, the approaches described herein can be used in connection with other types of implantable medical devices such as a pulse generator (e.g., pacemaker, defibrillator). Further, although the medical device 100 of FIG. 1 is shown as a medical device that is implanted into the patient 10, the approaches described herein can be used in connection with an external medical device such as a monitoring device that is coupled to a patient's skin or a mobile device that includes one or more sensors for detecting physiological data and/or that can process detected physiological data.

In the example of FIG. 1, the medical device 100 includes electrodes 102 and 104, which sense physiological signals (e.g., electrical cardiac activation signals) of the heart 12. The sensed physiological signals can be used to generate electrocardiogram (ECG) data such as ECG waveforms, which represent sensed cardiac electric activity over time. ECG waveforms can represent measured voltage amplitudes over time.

As described in more detail below, certain physiological data can be communicated (e.g., communicated wirelessly using an antenna) to a different component within the system. For example, physiological data can be communicated from the medical device 100 to a receiver 106, a computing system 108, and/or a remote computing device 110. The receiver 106 can be a device that is capable of programming, controlling, monitoring, and/or otherwise communicating with the medical device 100. The receiver 106 can help facilitate communication from the medical device 100 to another device or system such as the computing system 108 (e.g., laptop computer, desktop computer, server). The receiver 106 and/or the computing system 108 can be communicatively coupled to another computing system 110 with a display on which users (e.g., patients, physicians, technicians) can view data sensed and recorded by the medical device 100. In certain instances, the receiver 106 is a mobile computing device such as a programmer, smartphone, tablet computer, laptop computer, etc.

The medical device 100 can be programmed to initially store sensed ECG data to temporary memory such as a buffer or cache memory. But temporary memory can have limited storage capacity. To deal with the limited storage capacity, the medical device 100 can be programmed to cause physiological data to be repeatedly deleted as the amount of stored ECG data reaches the maximum storage capacity of the temporary memory. More specifically, the temporary memory can be operated as a first-in-first-out (FIFO) buffer or circular buffer that is operated to repeatedly delete the oldest data in favor of storing the newest data. However, under certain conditions, physiological data can be transferred and recorded to longer-term memory. Some or all of the physiological data recorded to longer-term memory can then be periodically transmitted to another device (e.g., the receiver 106, the computing system 108, and/or the remote computing device 110).

### Medical Devices

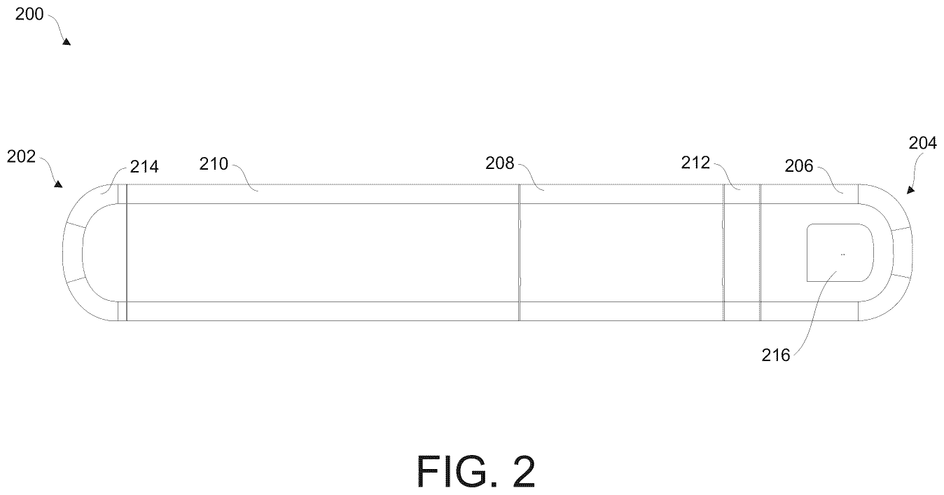

FIG. 2 is a side view of an implantable medical device 200 (hereinafter “IMD 200” for brevity). The IMD 200 may be, or may be similar to, the medical device 100 depicted in FIG. 1 and may be used in the system 100 of FIG. 1. Although the IMD 200 is implantable, other types of medical devices can include features described herein.

The IMD 200 includes an external housing that extends between a first end 202 and a second end 204. In the example of FIG. 2, the IMD 200 includes a first housing section 206, a second housing section 208, a third housing section 210, a fourth housing section 212, a first electrode 214, and a second electrode 216. Each of the housing sections can be separate components that are assembled together during manufacturing to create the external housing of the IMD 200. When assembled together, the housing sections can create a hermetically sealed enclosure. Although four separate housing sections are shown in FIG. 2, additional or fewer separate sections can be used to create the IMD 200. As will be described in more detail below, one or more of the housing sections can comprise a ceramic material.

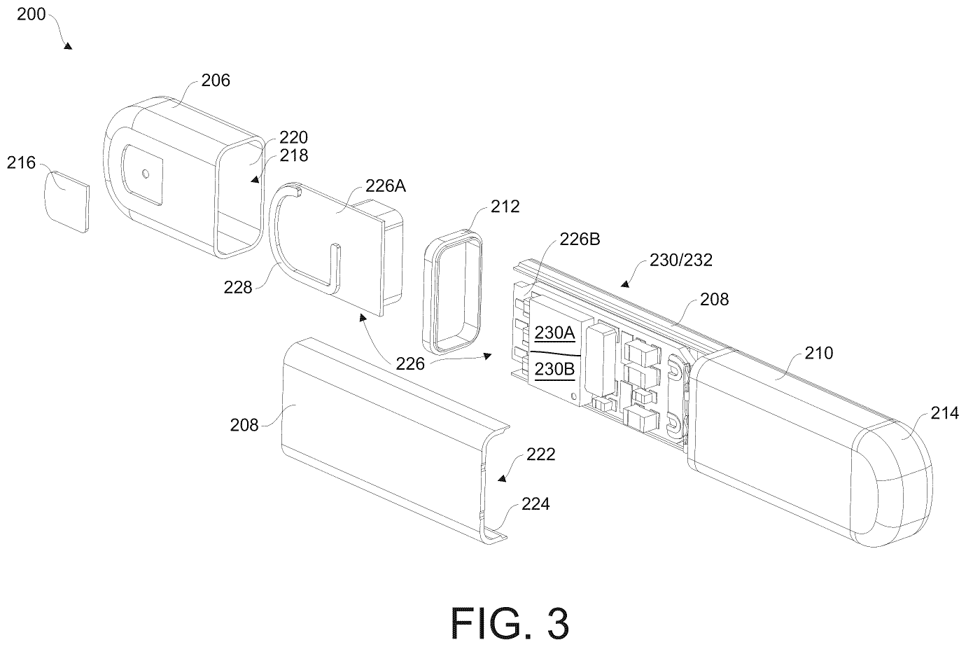

FIG. 3 shows a partially exploded view of the IMD 200. The first housing section 206 includes a first cavity 218 that is defined by one or more interior surfaces 220 of first housing section 206. The second electrode 216 is coupled to the first housing section 206. In certain instances, the first housing section 206 comprises a ceramic material.

The second housing section 208 includes a second cavity 222 that is defined by one or more interior surfaces 224 of second housing section 208. In FIG. 2, the second housing section 208 is shown as being comprised of multiple housing components. For example, the second housing section 208 (as with other housing sections) can be assembled from multiple components (e.g., welded together) to create its portion of the external housing of the IMD 200. In certain instances, the second housing section 208 comprises a metal material such as titanium. In other instances, the second housing section 208 comprises a ceramic material.

The third housing section 210 can comprise or can be a battery assembly (which may include one or more batteries). The exterior of the battery assembly can form the third housing section 210 in which one or more batteries (e.g., rechargeable battery cells) are positioned. The first electrode 214 is disposed at an end of the third housing section 210. In certain instances, the first electrode 214 is integrated with the battery assembly.

The fourth housing section 212 can function as an interface or coupler between the first housing section 206 and the second housing section 208. For example, the fourth housing section 212 can be used to couple the first housing section 206 to the second housing section 208. More specifically, in instances where the fourth housing section 212 comprises a metal such as titanium, the fourth housing section 212 can be assembled to the second housing section 208 via welding (e.g., laser welding). And, in instances where the first housing section 206 comprises a ceramic, the fourth housing section 212 can be brazed to the first housing section 206. In such instances, the fourth housing section 212 can be coupled between the first housing section 206 and the second housing section 208. As shown in FIG. 3, the fourth housing section 212 can be shaped as a continuous ring with an opening therethrough. Further, the fourth housing section 212 can include joint features such as one or more thinned sections or flange sections such that connecting the fourth housing section 212 to the other sections (e.g., via welding and/or brazing) can be accomplished. For example, portions of the other housing sections can overlap with the thinned or flanged sections of the fourth housing section 212 to provide overlapping surface area.

FIG. 3 shows a circuit board 226 with a first circuit board section 226A and a second circuit board section 226B. For purposes of illustrating the various components of the IMD 200 in an exploded view, the first circuit board section 226A and the second circuit board section 226B are shown as two separate components, but the two sections can form a single circuit board. The first circuit board section 226A and the second circuit board section 226B can comprise a rigid circuit board or a flexible circuit (e.g., a flexible circuit comprising polyimide). In instances where the first circuit board section 226A and the section circuit board section 226B are separate components, one section can comprise a rigid circuit board and the other section can comprise a flex circuit. Further, the two sections can be electrically coupled to each other.

When the IMD 200 is assembled, the first circuit board section 226A is positioned within the first cavity 218 and the second circuit board section 226B is positioned within the second cavity 222. Portions of either or both of the first circuit board section 226A and the second circuit board section 226B can extend through the fourth housing section 212 when the IMD 200 is assembled. As such, in instances with a single, continuous circuit board, the circuit board 226 can extend within the first cavity 218 and the second cavity 222 and through the opening of the fourth housing section 212.

An antenna 228 is positioned within the first cavity 218 and coupled to the circuit board 226 such as to the first circuit board section 226A. As one example, the antenna 228 can be embedded within the first circuit board section 226A. In this example, the antenna 228 can be formed by a conductive trace in the first circuit board section 226A. As another example, the antenna 228 can be formed on the interior surface 220 of the first housing section 206 and electrically coupled (e.g., directly coupled or indirectly coupled) to a conductive trace in the first circuit board section 226A. In this example, a portion of the interior surface 220 can be metalized if the interior surface 220 is otherwise a ceramic material. As another example, the antenna 228 can be embedded in the first housing section 206 and electrically coupled to a conductive trace in the first circuit board section 226A. In other instances, the antenna 228 can be positioned within the external housing between the third housing section and the first electrode 214 (e.g., positioned in a space between the battery and the first electrode 214). In other instances, the antenna 228 can be positioned outside the external housing.

Various other electrical components can be coupled to the circuit board 226 such as on the second circuit board section 226B. For example, the medical device 200 can include one or more memory components 230 coupled to the circuit board 226. The memory 230 can include a temporary memory 230A (e.g., random access memory (RAM), cache memory) and longer-term memory 230B (e.g., flash memory). In some instances, the temporary memory 230A and the longer-term memory 230B are separate sections or modules of a single chip package. In other instances, the temporary memory and the longer-term memory are separate components that are separately attached to the circuit board 226. In certain instances, the temporary memory 230A is operated as a memory (e.g., a FIFO buffer memory) that automatically deletes (or overwrites) old data as new data is stored to the temporary memory 230A-whereas the longer-term memory 230B is not operated to automatically delete (or overwrite) old data but, instead, manages its storage capacity according to the priority of physiological data already saved to the memory compared to the priority of new physiological data. Examples of how recording physiological data for certain cardiac events to the longer-term memory 230B can be prioritized over physiological data for other cardiac events. In certain instances, the longer-term memory 230B has a greater storage capacity (e.g., a greater amount of bytes, kilobytes, megabytes, gigabytes, and the like) than the storage capacity of the temporary memory 230A.

Other electrical components can be coupled to the circuit board 226 too such as one or more integrated circuits (e.g., application specific integrated circuits, field-programmable gate arrays) programmed to perform functions such as processing and/or communication functions of the IMD 200. For example, the electrical components like the integrated circuits can include one or more processors 232 (e.g., microprocessors) coupled to the memory 230 or other memory components with instructions (e.g., in the form of firmware, and/or software) for performing functions of the IMD 200. Example functions of the circuitry include processing physiological data (e.g., converting sensed electrical signals from the electrodes into ECG data, detecting potential cardiac events based on the sensed electrical signals, saving physiological data to the memory, and the like). The electrical components can be electrically coupled to the one or more batteries such that the electrical components are powered by the one or more batteries.

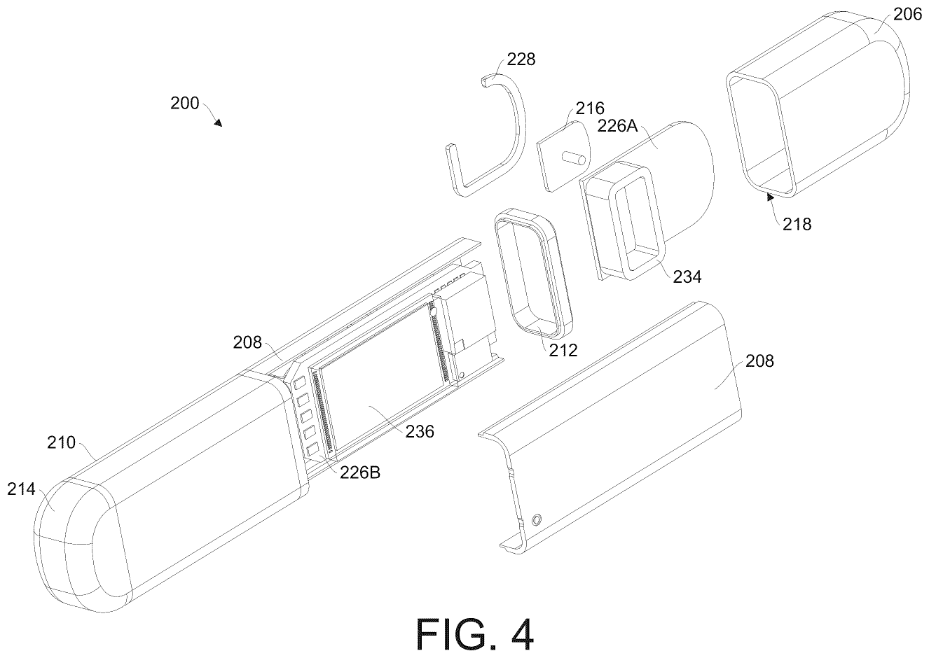

FIG. 4 shows another view of the IMD 200. As shown in FIG. 4, in certain instances, a charge coil 234 (e.g., a set of coils comprising a conductive material) is coupled to the circuit board 226 such as the first circuit board section 226A. The charge coil 234 is arranged to receive external signals (e.g., electromagnetic signals generated by a power transmitter) that induce a current in the charge coil 234 such that the current can recharge batteries in the battery assembly. When the IMD 200 is assembled, the charge coil 234 is positioned within the first cavity 218 of the first housing section 206 such that electromagnetic signals can pass through the housing and reach the charge coil 234. In certain instances, the charge coil 234 is wound such that there is a hole or space through the center of the charge coil 234 (e.g., the charge coil 234 has a donut shape or a similar shape). The charge coil 234 (and other electrical components) is electrically coupled to the one or more batteries 236 of the IMD 200.

### Methods for PVC Detection and Recording

As noted above, to save power and memory capacity, medical devices may not ultimately record to memory and transmit all the physiological data (e.g., ECG data) that was sensed by the medical devices. More specifically, although ECG data may be initially and temporarily stored to buffer memory or cache memory, most of the ECG can be permanently deleted (and not saved to longer-term memory) as the buffer/cache memory fills with newer ECG data. However, medical devices can be programmed to record and transmit certain physiological data associated with cardiac events such as PVC episodes, among other types of cardiac events.

But, even limiting recording and transmitting physiological data associated with PVCs can be an inefficient use of the medical device's power and memory capacity. Various approaches described herein can help determine which PVC episodes (and the underlying ECG data) should be prioritized for recording and transmission.

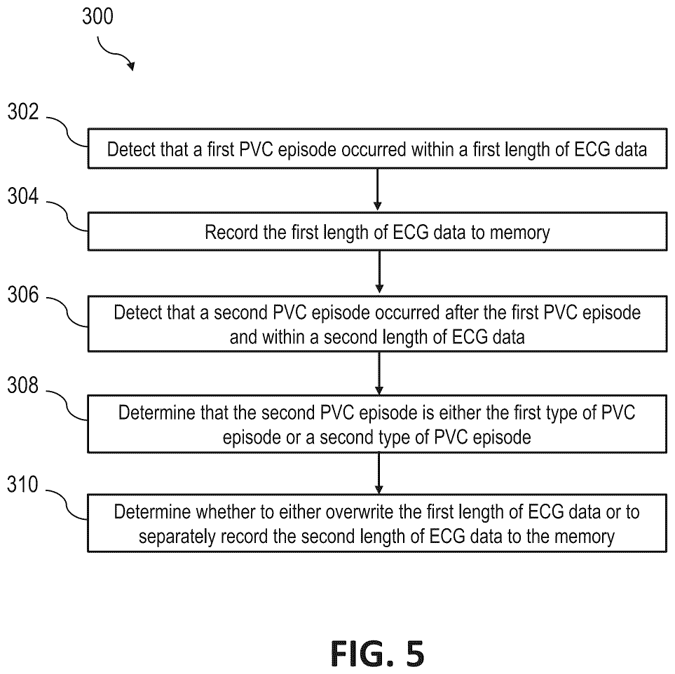

FIG. 5 shows an outline of steps of a method 300 for use with medical devices such as the medical devices 100 and 200 described herein. The method 300 is described below in the context of the medical device 200 of FIGS. 2-4, but the method 300 can be carried out by other medical devices.

The method 300 includes detecting that a first PVC episode occurred within a first length of ECG data (block 302 of FIG. 5). In certain instances, not all occurrences of a PVC result in a PVC episode being detected or otherwise determined to be ECG data with a PVC episode. For example, the medical device 200 may be programmed with a threshold value that must be met before a PVC episode is determined to have occurred. As examples, the threshold value may be a minimum number or minimum percentage of PVCs within a given time period. In certain instances, the minimum threshold is set as a minimum 5% PVC burden.

The medical device 200 can be programmed to distinguish between or classify different type of PVC episodes. For example, one type of PVC episode can be an episode with a maximum burden value over a given period of time (e.g., a time interval of approximately one minute or longer such as 1-2 minutes). Another type of PVC episode can be an episode with couplets (e.g., back-to-back PVCs) or triplets (e.g., back-to-back-to-back PVCs). Another type of PVC episode can be an episode with a particular morphology or shape.

Referring back to FIG. 5, the first PVC episode detected in block 302 can be associated with one or more types of PVC episodes. For example, the medical device 200 can be programmed to determine a maximum burden value of the detected PVC episode, whether the detected PVC episode included a couplet or triplet, and/or the morphologies of PVCs occurring during the PVC episode.

In response to detecting that a first PVC episode occurred, a length of the ECG data associated with the first PVC episode can be recorded to longer-term memory (block 304 in FIG. 5). For example, ECG data can be initially stored to temporary memory and, in response to detecting a PVC episode, a certain length of the ECG data can then be recorded to longer-term memory. The ECG data initially stored to the temporary memory can be deleted as it normally would have as newer ECG data is stored to the temporary memory (e.g., in a FIFO priority). In certain instances, the length (e.g., the amount of time) of ECG data recorded to the longer-term memory can depend on the occurrence of PVCs and the length of ECG data still available from the temporary memory. For example, the medical device 200 can identify or otherwise estimate the onset and ending of a PVC episode and record the ECG data between the onset and ending or record a predetermined amount (e.g., 10-60 seconds) of ECG data before the onset and after the ending of the PVC episode—to the extent the ECG data has not already been deleted from the temporary memory.

The method 300 further includes detecting that a second PVC episode occurred after the first PVC episode and within a second length of ECG data (block 306 in FIG. 5). As previously noted, the medical device 200 may be programmed with a threshold value that must be met before a PVC episode is determined to have occurred.

The method 300 includes determining that the second PVC episode is either the same type of episode as the first PVC episode or a different type of episode than the first PVC episode (block 308 in FIG. 5).

The method 300 further includes determining whether to either overwrite the first length of ECG data or to separately record the second length of ECG data to the memory (block 310 in FIG. 5). Determining whether to overwrite ECG data from the longer-term memory or separately record ECG data associated with a later PVC episode can depend on the type of the later PVC episode. When a later PVC episode is the same type as a prior PVC episode, the ECG data containing the prior PVC episode can be overwritten by ECG data containing the later PVC episode, in certain instances.

As one example, the maximum PVC burden values of the two PVC episodes can be compared to each other, and the PVC episode (and its ECG data) with the highest burden value can be recorded to the longer-term memory. If the prior PVC episode is associated with a higher PVC burden, then the later PVC episode can be ignored. However, if the later PVC episode has a higher burden than a PVC episode already stored to longer-term memory, the later PVC episode (and its ECG data) can overwrite the prior PVC episode (and its ECG data). If a subsequent PVC episode has an even higher burden, the subsequent PVC episode can overwrite a prior PVC episode.

Alternatively, instead of recording a single PVC episode with the highest PVC burden, the medical device 200 can be programmed to maintain a maximum number of representative PVC episodes (e.g., ECG data from PVC episodes with the three highest PVC burdens). As another alternative, the medical device 200 could be programmed to record examples of ECG data associated with different PVC burden values such as one or more representative PVC episodes having a burden value of 5-10%, one or more representative PVC episodes having a burden value of 10-20%, one or more representative PVC episodes having a burden value of greater than 20%, and so on. Each of the burden value ranges can be considered to be a different bucket, and a single representative PVC episode can be assigned to each bucket—to the extent a representative episode has been detected for a given bucket.

Using the approaches described above, the ECG data with the highest PVC burden values is prioritized over PVC episodes with lower PVC burden values. This approach can save power and memory resources of the medical device 200 because certain PVC episodes (e.g., those with lower PVC burden values) can be ignored and not recorded to longer-term memory to limit consumption of power and memory capacity.

As another example of overwriting prior PVC episodes, a similar approach can be used with PVC episodes involving couplet events and/or triplet events. The medical device 200 can be programmed to determine the number of couplets and/or triplets occurring during PVC episodes. In certain instances, a minimum number of couplet events or triplet events must occur in a given period of time before the medical device 200 determines that a PVC episode has occurred. When the medical device 200 detects a couplet event and/or a triplet event, the medical device 200 can determine whether ECG data from the same type of event has already been recorded to longer-term memory and, if so, whether the current PVC episode includes the same, fewer, or more couplets and/or triplets. If the current PVC episode has more couplets and/or triplets, the ECG data of the current PVC episode can be written over the ECG data of the prior PVC episode.

In certain instances, overwriting prior PVC episodes recorded to longer-term memory with later PVC episodes involves recording over the previously recorded ECG data, deleting the previously recorded ECG data before writing the new ECG data, and/or separately recording a later PVC episode to the longer-term memory but associating the previously recorded ECG data with a flag or indicator that prevents the previously recorded ECG data from being transmitted by the medical device 200 to another device such that the later PVC episode is transmitted instead of the prior PVC episode.

One optional exception to the overwriting approaches described herein is that the medical device 200 can be programmed to record to longer-term memory (and prevent overwriting) ECG data that is associated with the first PVC episode detected during a given period of time. More specifically, the medical device 200 may be programmed to transmit any ECG data recorded to longer-term memory once a day, twice a day (e.g., daytime, nighttime), once every 4-12 hours (e.g., 4 hours, 8 hours, 12 hours), once in communication with a receiver, or some other time period or trigger (e.g., in response to a command). After the recorded ECG data is transmitted to a different device, the prior recorded ECG data can be deleted or otherwise marked for deletion, and the medical device 200 can restart by detecting and recording new PVC episode for the new time period. The first PVC episode detected (e.g., the first PVC episode that meets a minimum threshold) can be recorded to the longer-term memory, and the medical device 200 can prevent that first PVC episode from being overwritten by subsequent PVC episodes regardless of whether subsequent PVC episodes have higher PVC burden values, couplet occurrences, triplet occurrences, etc.

When a later PVC episode is a different type than the prior PVC episode, the ECG data containing the later PVC episode can be separately written to the longer-term memory such that both the prior and the later PVC episodes are recorded to the longer-term memory. For example, the longer-term memory can separately store at least one PVC episode with a couplet event, another PVC episode with a triplet event, another PVC episode with the maximum PVC burden, another PVC episode that represents a median PVC burden, and another PVC episode that was the first PVC episode detected by the medical device 200. In certain instances, the number of PVC episodes can be customized on a per-patient basis. For example, medical devices used with patients with lower average or median PVC burden values (e.g., ˜10%) can have a lower maximum for the number of PVC episodes (e.g., 3 episodes) stored to longer-term memory compared to patients with a higher average or median PVC burden values (e.g., ˜25%), which may have a maximum number such as 5 episodes. Such maximums can be updated automatically by the medical device 200 based on historical PVC data or may be a result of reprogramming the medical device 200.

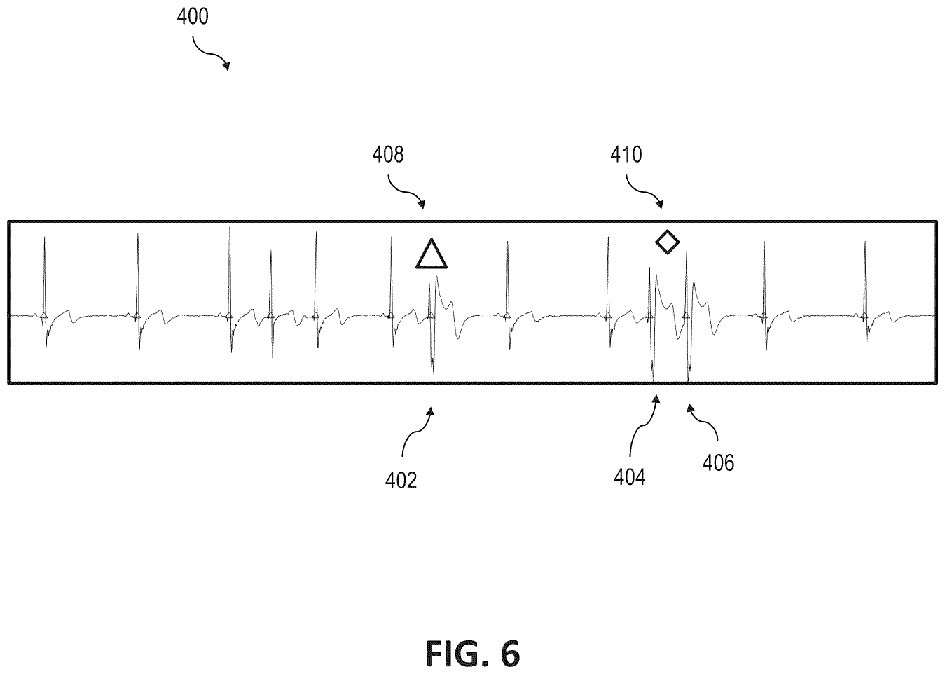

In addition to detecting the different types of PVC episodes described above, the medical device 200 can be programmed to detect different morphologies or shapes of PVCs. FIG. 6 shows an example of a short strip 400 of ECG data that contains three PVCs 402, 404, and 406. PVCs 404 and 406 occurred back-to-back (e.g., without a normal heartbeat between the PVCs) and are an example of a PVC couplet. The shapes of PVCs can be analyzed by the medical device 200 to determine whether a given PVC episode contains similar-shaped or different-shaped PVCs. If the medical device 200 detects a PVC episode (e.g., by meeting a minimum threshold) and the medical device 200 detects two or more different shapes or morphologies of PVCs, the ECG data associated with the PVC episode can be recorded to the longer-term memory.

As noted above, the medical device 200 may be programmed to transmit (e.g., wirelessly transmit) any ECG data recorded to longer-term memory periodically to another device such as a receiver. The ECG data may ultimately be transmitted to a clinic for review by a physician.

To assist with the review, metadata can be generated and used to denote detected PVCs when the strip 400 is displayed. For example, single PVCs can be denoted by a first type 408 of annotation, couplets can be denoted by a second type 410 of annotation, and so on. In other instances, the same annotation shape/approach is used for all PVCs but repeated for each individual PVC (e.g., a couplet of PVCs would be associated with two separate annotations—one for each individual PVC).

In certain instances, before the ECG data is transmitted to a clinic, the ECG data is transmitted and processed by a computing system such as a server. Because the server has more computational and memory capacity than a medical device, the server can provide a second review of the ECG data before clinical review. The server can be programmed to filter out false PVC episodes and noise, to confirm PVC episodes, to cluster together similar shaped PVCs, to determine which PVC episode warrants highest priority, and/or to determine confidence values for individual PVCs and PVC episodes to prioritize certain PVC episodes over other episodes, among other functions.

To assist with the functions described immediately above, the server can include one or more machine learning models (e.g., types of deep neural networks) to process the ECG data to classify cardiac activity. For example, the machine learning model may compare the ECG data to labeled ECG data to determine which labeled ECG data the ECG data most closely resembles. The labeled ECG data may identify a particular cardiac event such as a PVC episode. In certain instances, the machine learning model includes two paths, where the first path is a deep convolutional neural network and the second path is a deep fully-connected neural network. The deep convolutional neural network receives one or more sets of beats (e.g., beat trains with 3-10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, and a non-linear activation function layer. This set of layers can be repeated multiple times.

The deep fully connected neural network can receive RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

If the machine learning model determines that the ECG data most closely resembles a labeled ECG data associated with a cardiac event, then the machine learning model may determine that the patient has experienced that cardiac event. Additionally, the machine learning model may measure or determine certain characteristics of the cardiac activity of the patient based on the ECG data. For example, the machine learning model may determine a heart rate, a duration, or a beat count of the patient during the cardiac event based on the ECG data. The server stores the cardiac event and associated metadata such as information like beat classification, heart rate, duration, beat count, etc., in a database for storage.

Outputs of the machine learning model and the underlying ECG data can be transmitted to a clinic for physician review.

### Computing Devices and Systems

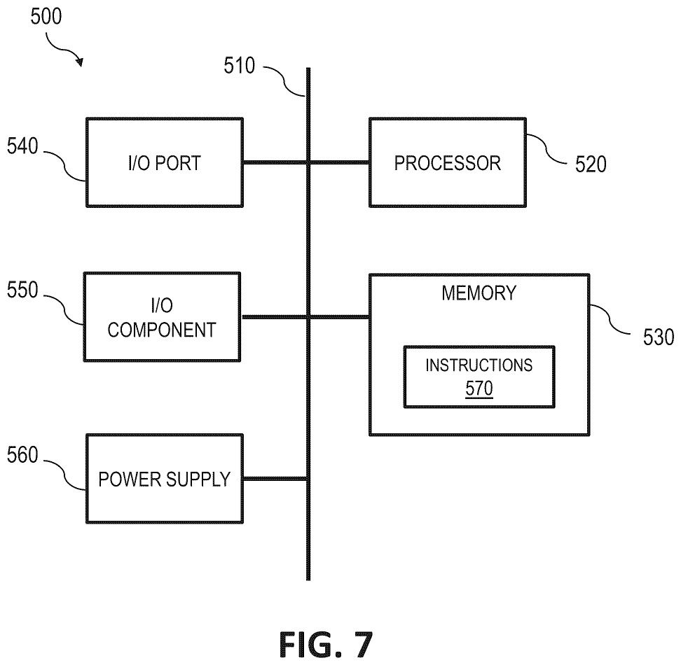

FIG. 7 is a block diagram depicting an illustrative computing device 500, in accordance with instances of the disclosure. The computing device 500 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 7 and described below. For example, the medical devices, receivers, and computing systems can each include their own set (or partial set) of components shown in FIG. 7 and described below.

In instances, the computing device 500 includes a bus 510 that, directly and/or indirectly, couples one or more of the following devices: a processor 520, a memory 530, an input/output (I/O) port 540, an I/O component 550, and a power supply 560. Any number of additional components, different components, and/or combinations of components may also be included in the computing device 500.

The bus 510 represents what may be one or more busses (such as, for example, an address bus, data bus, or combination thereof). Similarly, in instances, the computing device 500 may include a number of processors 520, a number of memory components 530, a number of I/O ports 540, a number of I/O components 550, and/or a number of power supplies 560. Additionally, any number of these components, or combinations thereof, may be distributed and/or duplicated across a number of computing devices.

In instances, the memory 530 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 530 stores computer-executable instructions 570 for causing the processor 520 to implement aspects of instances of components discussed herein and/or to perform aspects of instances of methods and procedures discussed herein. The memory 530 can comprise a non-transitory computer readable medium storing the computer-executable instructions 570.

The computer-executable instructions 570 may include, for example, computer code, machine-useable instructions, and the like such as, for example, program components capable of being executed by one or more processors 520 (e.g., microprocessors) associated with the computing device 500. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 570 may be configured to be executed by the processor 520 and, upon execution, to cause the processor 520 to perform certain processes. In certain instances, the processor 520, memory 530, and instructions 570 are part of a controller such as an application specific integrated circuit (ASIC), field-programmable gate array (FPGA), and/or the like. Such devices can be used to carry out the functions and steps described herein.

The I/O component 550 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical device comprising:
a housing;
electrodes coupled to the housing and for sensing cardiac activation signals used to generate electrocardiogram (ECG) data;
memory; and
one or more processors programmed with instructions to cause the medical device to:
detect that a first premature ventricular contraction (PVC) episode occurred within a first length of ECG data, wherein the first PVC episode is a first type of PVC episode,
in response to detecting the first PVC episode, record the first length of ECG data to the memory,
detect that a second PVC episode occurred after the first PVC episode and within a second length of ECG data,
determine that the second PVC episode is either the first type of PVC episode or a second type of PVC episode, and
determine whether to either overwrite the first length of ECG data or to separately record the second length of ECG data to the memory based, at least in part, on the second PVC episode being the first type of PVC episode or the second type of PVC episode.

2. The medical device of claim 1, further comprising:
a buffer, wherein the first length of ECG data is temporarily stored to the buffer before being recorded to the memory.

3. The medical device of claim 2, wherein the ECG data temporarily stored to the buffer but not recorded to the memory is permanently deleted.

4. The medical device of claim 1, wherein the one or more processors are programmed with the instructions to further cause the medical device to:
determine that the second PVC episode is the first type of PVC episode, and
in response, overwrite the first length of ECG data with the second length of ECG data.

5. The medical device of claim 1, wherein the first type of PVC episode and the second type of PVC episode are maximum burden episodes, wherein the first PVC episode is associated with a first burden value, wherein the second PVC episode is associated with a second burden value, wherein the first length of ECG data is overwritten by the second length of ECG data in response to the second burden value being greater than the first burden value.

6. The medical device of claim 5, wherein the first burden value and the second burden value are based on a number of PVCs within a predetermined time period.

7. The medical device of claim 1, wherein the first type of PVC episode and the second type of PVC episode are couplet events and/or triplet events, wherein the first PVC episode is associated with a first number of couplets or triplets, wherein the second PVC episode is associated with a second number of couplets or triplets, wherein the first length of ECG data is overwritten by the second length of ECG data in response to the second number of couplets or triplets being greater than the first number of couplets or triplets.

8. The medical device of claim 1, wherein the first PVC episode is detected based, at least in part, on a minimum PVC threshold being met.

9. The medical device of claim 1, wherein the one or more processors are programmed with the instructions to further cause the medical device to:
wirelessly transmit, at periodic intervals, any PVC episodes recorded to the memory to a remote computing device.

10. The medical device of claim 1, wherein the memory comprises a longer-term memory and a temporary memory, wherein the first length of ECG data is first recorded to the temporary memory and then to the longer-term memory.

11. The medical device of claim 10, wherein the longer-term memory has a greater storage capacity than the storage capacity of the temporary memory.

12. The medical device of claim 10, wherein the longer-term memory is flash memory, wherein the temporary memory is random-access memory.

13. The medical device of claim 10, wherein the temporary memory is a first-in first-out (FIFO) memory.

14. A method comprising:
detecting that a first premature ventricular contraction (PVC) episode occurred within a first length of electrocardiogram (ECG) data, wherein the first PVC episode is a first type of PVC episode;
in response to the detecting, recording the first length of ECG data to memory;
detecting that a second PVC episode occurred after the first PVC episode and within a second length of ECG data;
determining that the second PVC episode is either the first type of PVC episode or a second type of PVC episode; and
determining whether to either overwrite the first length of ECG data or to separately record the second length of ECG data to the memory based, at least in part, on the determining that the second PVC episode is the first type of PVC episode or the second type of PVC episode.

15. The method of claim 14, further comprising:
determining that the second PVC episode is the first type of PVC episode; and
in response, overwriting the first length of ECG data with the second length of ECG data.

16. The method of claim 14, wherein the first type of PVC episode and the second type of PVC episode are maximum burden episodes, wherein the first PVC episode is associated with a first burden value, wherein the second PVC episode is associated with a second burden value, wherein the first length of ECG data is overwritten by the second length of ECG data in response to the second burden value being greater than the first burden value.

17. The method of claim 16, wherein the first burden value and the second burden value are based on a number of PVCs within a predetermined time period.

18. The method of claim 14, wherein the first type of PVC episode and the second type of PVC episode are couplet events and/or triplet events, wherein the first PVC episode is associated with a first number of couplets or triplets, wherein the second PVC episode is associated with a second number of couplets or triplets, wherein the first length of ECG data is overwritten by the second length of ECG data in response to the second number of couplets or triplets being greater than the first number of couplets or triplets.

19. The method of claim 14, wherein the first type and the second type are different types of PVC episodes, the method further comprising:
determining that the second PVC episode is the second type of PVC episode; and
in response, recording the second length of ECG data to the memory such that the memory contains both the first length of ECG data and the second length of ECG data.

20. The method of claim 19, wherein the first type of PVC episode is a maximum burden episode, wherein the second type of PVC episode is a couplet episode or a triplet episode.
